(19) United States
(12) Patent Application Publication
(10) Pub. No.: US 2021/0127647 A1
(43) Pub. Date: May 6, 2021

(54) Non-Human Animal Model of Non-Alcoholic Fatty Liver Disease

(71) Applicant: The Nippon Dental University, Tokyo (JP)

(72) Inventors: Ken Yaegaki, Tokyo (JP); Hiroshi Ishikawa, Tokyo (JP)

(21) Appl. No.: 16/641,290

(22) PCT Filed: Aug. 25, 2017
(86) PCT No.: PCT/JP2017/030498

§ 371 Date: Dec. 28, 2020

(51) Int. Cl. A01K 67/027; A23K 50/30

(52) U.S. Cl. CPC A01K 67/027; A23K 50/30; A01K 2207/20; A01K 2267/03; A01K 2207/25; A01K 2227/108

(57) ABSTRACT

Provided is a non-human model animal for non-alcoholic fatty liver disease (NAFLD) (NAFLD model animal) which reflects clinical manifestations in humans. The non-human model animal for non-alcoholic fatty liver disease (NAFLD) is prepared by rearing a non-human animal by feeding the animal with a choline-deficient L-amino acid-defined (CDAA) diet which is substantially free of protein.

### TECHNICAL FIELD

The present invention relates to a non-human model animal for non-alcoholic fatty liver disease (NAFLD), and a method of preparing the animal.

### BACKGROUND ART

Non-alcoholic fatty liver disease (NAFLD), whose basic lesion is fatty liver, is a disease state which exhibits tissue changes of the hepatic parenchyma such as inflammation, necrosis, and fibrosis that are similar to those in alcoholic liver injury, even without a long history of alcohol intake. NAFLD is basically asymptomatic. During the course of progression of the disease state, the fatty liver leads to steatohepatitis, liver cirrhosis, and then to liver cancer. The steatohepatitis in NAFLD is called non-alcoholic steatohepatitis (NASH). In particular, in recent years, metabolic syndromes due to obesity, diabetes, and/or the like have become a social problem, and NASH is also regarded as one of the metabolic syndromes. As complications of NAFLD and NASH, lifestyle-related diseases such as obesity, diabetes, hyperlipidemia, and hypertension are found, and their major clinical manifestations characteristically include increases in the blood alanine aminotransferase (ALT) and hyaluronic acid levels, and on the other hand, decreases in the total blood cholesterol and albumin levels. However, the pathogenic mechanisms of NAFLD and NASH still remain largely unclear, and there is no established effective therapeutic method or therapeutic agent therefor at present. This is partly due to the fact that, since development of NAFLD or NASH is based on lifestyle-related diseases of humans, non-human model animals suitable for research of NAFLD and NASH have not been established yet.

Elucidation of the disease states of NAFLD and NASH, which may progress to fatal diseases such as liver cirrhosis and liver cancer, is indispensable for development of effective therapeutic methods and therapeutic agents therefor, and the elucidation requires appropriate non-human model animals for NAFLD and NASH.

Model animals for NASH have so far been reported (for example, Patent Documents 1 and 2). However, non-human model animals for NAFLD, and also for NASH regarding large animals, have been hardly reported. Under such circumstances, a non-human model animal for NAFLD, which model animal provides a better basis from the viewpoint of progression of the disease, is demanded. It can be said that, for the purpose of establishment of a therapeutic method and therapeutic agent for NAFLD in humans, an NAFLD model of a non-human animal that is physiologically, anatomically, and genetically closer to humans is particularly demanded.

### PRIOR ART DOCUMENTS

### Patent Documents

### Patent Document 1: WO 2011/013247

### Patent Document 2: WO 2008/001614

### SUMMARY OF THE INVENTION

In view of this, an object of the present invention is to provide a non-human model animal for NAFLD, a method of preparing the animal, and a diet to be used for the preparation.

In order to solve the above problem, the present inventors intensively studied. By feeding a micromini pig with a choline-deficient L-amino acid-defined (CDAA) diet having a particular composition, the present inventors successfully prepared an NAFLD model animal (model pig) from the micromini pig, which is physiologically, anatomically, and genetically closer to humans, thereby completing the present invention. The model pig showed changes in blood parameters characteristic to NAFLD, such as decreases in the total blood cholesterol and albumin levels, increases in the alanine aminotransferase (ALT) and aspartate aminotransferase (AST) levels, and a decrease in the hepaplastin test (HPT) value.

More specifically, specific examples of the present invention include the following.

[1] A method of preparing a non-human model animal for non-alcoholic fatty liver disease (NAFLD), the method comprising the step of rearing a non-human animal by feeding the animal with a choline-deficient L-amino acid-defined (CDAA) diet,

wherein the CDAA diet is substantially free of protein.

[2] The method according to [1], wherein the non-human animal is an animal belonging to a mammalian order selected from the group consisting of Primates, Rodentia, Carnivora, and Artiodactyla.
[3] The method according to [1] or [2], wherein the non-human animal is a pig.
[4] A non-human model animal for non-alcoholic fatty liver disease (NAFLD) prepared by the method according to any one of [1] to [3].
[5] A non-human animal model for non-alcoholic fatty liver disease (NAFLD), which exhibits at least one of the following findings (1) to (6):

(1) a decrease in the total blood cholesterol level;

(2) a decrease in the blood albumin level;

(3) a decrease in the hepaplastin test (HPT) value;

(4) an increase in the blood type IV collagen level;

(5) an increase in the blood alanine aminotransferase (ALT) level; and

(6) an increase in the blood hyaluronic acid level.

[6] A choline-deficient L-amino acid-defined (CDAA) diet which is substantially free of protein, to be used for causing non-alcoholic fatty liver disease (NAFLD) in a non-human animal.

The present invention provides a non-human model animal for non-alcoholic fatty liver disease (NAFLD), which non-human model animal is physiologically, anatomically, and genetically closer to humans, and a method of preparing the animal. The present invention also provides a diet to be used for causing non-alcoholic fatty liver disease (NAFLD) in a non-human animal.

### BRIEF DESCRIPTION OF THE DRAWING

FIG. 1 is a biopsy photograph of a liver tissue of a model animal (micromini pig) for non-alcoholic fatty liver disease (NAFLD) obtained by the method of the present invention.

### MODE FOR CARRYING OUT THE INVENTION

Provided is a non-human model animal that develops non-alcoholic fatty liver disease (NAFLD) (hereinafter also referred to as “model animal of the present invention”).

The model animal of the present invention can be prepared by rearing a non-human animal by feeding the animal with the later-mentioned choline-deficient L-amino acid-defined (CDAA) diet having a particular composition.

The non-human animal used for preparing the model animal of the present invention (hereinafter also referred to as “non-human animal used in the present invention”) is not limited as long as it is a non-human animal commonly used as an experimental animal. The non-human animal is preferably a non-human vertebrate, more preferably a non-human mammal, still more preferably an artiodactyl. Specific examples of the non-human animal used in the present invention include pigs, mini pigs, and micromini pigs. The non-human animal used in the present invention is preferably a mini pig or micromini pig, especially preferably a micromini pig. Examples of commercially available micromini pigs include micromini pigs manufactured by Fuji Micra Inc. (strain: Fuji Micromini Pig).

The non-human animal used in the present invention may be those suffering from lifestyle-related diseases such as obesity, diabetes, hyperlipidemia, and hypertension, similarly to humans. In cases where the non-human animal used in the present invention is suffering from a lifestyle-related disease(s), the lifestyle-related disease(s) in the non-human animal may be either a single kind of disease or a plurality of kinds of diseases. Each of these lifestyle-related diseases may be either a genetic congenital disease or an acquired disease.

The choline-deficient L-amino acid-defined (CDAA) diet used in the present invention (hereinafter also referred to as “diet used in the present invention”) is characterized in that it is substantially free of protein. More specifically, the term “substantially free of protein” means that the protein content with respect to the entire diet is preferably less than 1×10⁻⁷% by weight, more preferably less than 1×10⁻⁸% by weight, especially preferably 0% by weight.

The composition of the diet used in the present invention is not limited as long as it satisfies the protein content described above, and as long as it allows induction of symptoms of NAFLD in the non-human animal used in the present invention. The composition may be set appropriately depending on, for example, the type of the non-human animal used in the present invention. For example, the composition of the diet used in the present invention may be set based on the composition of a conventional choline-deficient L-amino acid-defined (CDAA) diet. The setting of the composition of the diet of the present invention and preparation of the diet can be easily carried out by those skilled in the art by referring to, for example, descriptions in the present description, or known information or technique such as A15022101 and A02082002B manufactured by Research Diets, Inc. (US). The diet used in the present invention may be prepared based on known information or technique, or may be prepared by practical application or improvement of the commercially available products.

In the method of the present invention, the time when feeding of the non-human animal used in the present invention with the CDAA diet used in the present invention is begun is not limited as long as induction of NAFLD is possible, and may be appropriately set depending on, for example, the type of the non-human animal and/or the presence or absence of complications. For example, in cases where the non-human animal used in the present invention is suffering from a lifestyle-related disease such as diabetes, the feeding is preferably begun after the development of the lifestyle-related disease. The amount of the diet to be ingested is not limited as long as NAFLD can be induced. The amount may be appropriately set depending on, for example, the type of the non-human animal used in the present invention, and/or the size or body weight thereof. In cases where, for example, a micromini pig is used, the amount of the diet to be ingested is about 300 g per day. The period during which the animal is reared by feeding with the diet is not limited as long as NAFLD can be induced. The period may be appropriately set depending on, for example, the type of the non-human animal used in the present invention. In cases where, for example, a micromini pig is used, the micromini pig is reared by feeding with the diet for usually not less than 6 weeks, preferably not less than 8 weeks, more preferably not less than 12 weeks. By rearing the animal using the diet used in the present invention, non-alcoholic fatty liver disease (NAFLD) can be induced. In cases where, for example, a micromini pig is used, its rearing by continuous feeding with the diet may lead to accumulation of ascites fluid at about Week 14 or later, resulting in death.

The “rearing by feeding with the diet” in the method of the present invention includes not only cases where the animal is fed with the CDAA diet used in the present invention, but also cases where the animal is fed with separate components in the CDAA diet. As long as NAFLD can be induced, other diets may also be used in combination.

The NAFLD model animal that can be prepared by the method of the present invention well reflects the clinical manifestations characteristic to NAFLD, especially human NAFLD, and exhibits slow progression of the disease state similarly to humans with NAFLD. Thus, the NAFLD model animal that can be prepared by the method of the present invention is useful because it can be readily extrapolated to humans. Moreover, the model animal can be easily prepared because development of NAFLD symptoms can be caused simply by feeding the animal with the diet.

The model animal of the present invention exhibits at least one finding selected from the following (1) to (6):

(1) a decrease in the total blood cholesterol level;

(2) a decrease in the blood albumin level;

(3) a decrease in the hepaplastin test (HPT) value;

(4) an increase in the blood type IV collagen level;

(5) an increase in the blood alanine aminotransferase (ALT) level; and

(6) an increase in the blood hyaluronic acid level.

In the present description, the findings (1) to (6) may be collectively referred to as “NAFLD findings”.

In the findings (1) to (6), the “decrease” or “increase” means that the parameter corresponding each finding is “decreased” or “increased” relative to the “standard value” in a normal individual reared using a diet that does not cause NAFLD. More specifically, each finding may be evaluated based on a statistically significant difference in comparison with the “standard value”. For each finding, a known statistical analysis method may be appropriately selected and employed. Examples of the statistical analysis method include ANOVA in cases of a test among multiple groups, but, in the present invention, Bonferroni's multiple comparison is preferably used. In cases where the P-value is less than 0.05 in the test, it is judged that a “decrease” or an “increase” is found. On the other hand, in cases where the P-value is not less than 0.05, it can be judged that no “decrease” or “increase” is found. Examples of the diet that does not cause NAFLD, that is, a normal diet, include a solid feed MP-A (manufactured by Oriental Yeast Co., Ltd.) Among those skilled in the art such as sellers of experimental animals and researchers, it has been commonly recognized that there is no NAFLD finding or disease state in animals reared using a normal diet. It can thus be said that study of significance of the amount of change with time in the parameter corresponding to each of the above findings is especially preferred for preparation of the model animal of the present invention.

### EXAMPLES

The present invention is described below in more detail by way of Examples. However, the present invention is not limited by these Examples, and may be modified in various ways within the scope of the present invention.

### Example 1: Preparation of Non-Human Model Animal for NAFLD

Using micromini pigs, non-human model animals for NAFLD were prepared by the following procedure.

About 7-month-old male micromini pigs (body weight, about 10 kg) (strain: Fuji Micromini Pig) were purchased from Fuji Micra Inc., and six individuals of the pigs were acclimated to a rearing environment with natural ventilation at a temperature of 20±5° C. and a relative humidity of 55±25% under a light-dark cycle of 7:00 to 19:00 (light)/19:00 to 7:00 (dark) while being fed with 300 g/day of a solid diet MP-A (manufactured by Oriental Yeast Co., Ltd.; containing 0.29% choline and 15.3% crude protein), which is a normal diet. The feeding of the pigs was carried out at about 8:00 every day.

Table 1 shows the composition of the major components of the normal diet MP-A used.

TABLE 1

| | | MP-A |
|---|---|---|
| Component | | g(per 100 g) |
| | Calcium | 2.64 |
| | Phosphorus | 1.99 |
| | Magnesium | 0.2 |
| | Sodium | 0.51 |
| | Potassium | 2.43 |
| | Iron | 0.0292 |
| | Aluminum | 0.0121 |
| | Copper | 0.0009 |
| | Zinc | 0.016 |
| | Cobalt | 0.0003 |
| | Manganese | 0.00632 |
| Amino acid composition | Isoleucine | 0.53 |
| | Leucine | 1.01 |
| | Lysine | 0.70 |
| | Methionine | 0.24 |
| | Cystine | 0.22 |
| | Phenylalanine | 0.63 |
| | Tyrosine | 0.43 |
| | Threonine | 0.53 |
| | Tryptophan | 0.18 |
| | Valine | 0.68 |
| | Arginine | 0.76 |
| | Histidine | 0.37 |
| | Alanine | 0.72 |
| | Asparatic acid | 1.27 |
| | Glutamic acid | 2.18 |
| | Glycine | 0.82 |
| | Proline | 0.94 |
| | Serine | 0.65 |

| | | |
|---|---|---|
| Vitamin A | 1830 | IU |
| Vitamin D3 | 400 | IU |
| Vitamin E | 18.2 | mf |
| Vitamin B1 | 3.24 | mf |
| Vitamin B2 | 2.59 | mf |
| Vitamin B6 | 0.79 | mf |
| Vitamin 12 | 12 | μg |
| Vitamin C | 21 | mg |
| Niacin | 8.18 | mg |
| Pantothenic acid | 3.61 | mg |
| Biotin | 22.2 | μg |
| Folic acid | 0.20 | mg |
| Inositol | 226 | mg |

| | |
|---|---|
| Choline | 0.29% |

After an acclimation period of about 1 week, the pigs were fed ad libitum with 300 g/day of a CDAA diet (manufactured by Research Diets, Inc.; A15022101), which is substantially free of protein, in the same rearing environment for 16 weeks. The feeding of the pigs was carried out at about 8:00 every day.

Table 2 shows the composition of the major components of the CDAA diet used.

TABLE 2

| | A15022101 |
|---|---|
| Component | g(per 100 g) |
| Casein | 0 |
| Soy protein | 0 |
| DL-Methionine | 0 |
| L-Cystine | 0.32 |
| L-Isoleucine | 0.70 |
| L-Leucine | 1.31 |
| L-Lysine | 1.06 |
| L-Methionine | 0.11 |
| L-Phenylalanine | 0.76 |
| L-Threonine | 0.61 |
| L-Tryptophan | 0.20 |
| L-Valine | 0.79 |
| L-Histidine-HCl—H | 0.40 |
| L-Alanine | 0.54 |
| L-Arginine | 0.83 |
| L-Aspartic acid | 1.38 |
| L-Glutamic acid | 3.12 |
| Glycine | 0.48 |
| L-Proline | 1.19 |
| L-Serine | 0.84 |
| L-Trypsin | 0.70 |
| Corn starch | 9.25 |
| Maltodextrin 1 | 6.53 |
| Fructose | 23.9 |
| Sucrose | 1.09 |
| Cellulose, BW200 | 10.9 |
| Lard | 2.07 |
| Coconut oil | 2.39 |
| Primex | 18.5 |
| Mixed salt, S01004 | 5.44 |
| Sodium bicarbonate | 0.78 |
| Mixed vitamins, V0100 | 1.09 |
| Choline hydrogen tartrate | 0 |
| Cholesterol | 2.05 |
| Sodium cholate | 0.71 |
| FD&C yellow dye #5 | 0.005 |

As described later in detail, by the rearing of the pigs by feeding with the CDAA diet substantially free of protein, NAFLD micromini pigs which can be extrapolated to NAFLD were successfully prepared.

### Example 2: Study of Clinical Manifestations of Non-Human Model Animal for NAFLD

Micromini pigs reared as described above were studied in terms of clinical manifestations of NAFLD. As the clinical manifestations of NAFLD, the following items (1) to (6) (NAFLD findings) were set referring to indices for diagnosis of human NAFLD:

(1) a decrease in the total blood cholesterol level;

(2) a decrease in the blood albumin level;

(3) a decrease in the hepaplastin test (HPT) value;

(4) an increase in the blood type IV collagen level;

(5) an increase in the blood alanine aminotransferase (ALT) level; and

(6) an increase in the blood hyaluronic acid level.

### (a) Measurement Method

During the acclimation period of about 1 week with the normal diet MP-A, which is described in Example 1, no NAFLD disease state was found. Thus, from the 6 individuals of micromini pigs after the acclimation period, blood was collected to obtain serum, and the serum samples obtained were subjected to measurement of the following items (1) to (6). The measured values were regarded as “standard values”.

(1) Total blood cholesterol level

(2) Blood albumin level

(3) Hepaplastin test (HPT) value

(4) Blood type IV collagen level

(5) Blood alanine aminotransferase (ALT) level

(6) Blood hyaluronic acid level

Each of the items (1) to (6) was measured using a method, kit, or the like described below.

(1) The total blood cholesterol level was measured by the cholesterol dehydrogenase (UV) method.

(2) The blood albumin level was measured by nephelometry (the modified BCP method).

(3) The hepaplastin test (HPT) value was measured by coagulation time measurement.

(4) The blood type IV collagen level was measured using a Type IV collagen ELISA kit, ACB (manufactured by Funakoshi Corporation).

(5) The blood alanine aminotransferase (ALT) level was measured by the JSCC reference method.

(6) The blood hyaluronic acid level was measured by the latex agglutination turbidimetric immunoassay.

Subsequently, the six individuals of micromini pigs after the acclimation period were further reared using a CDAA diet (manufactured by Research Diets, Inc.; A15022101), which is substantially free of protein. Blood was collected every 2 weeks to obtain serum during the 16 weeks after the beginning of feeding with the CDAA diet substantially free of protein. Using the serum samples obtained, the above items (1) to (6) were measured.

### (b) Measurement Results

The standard values, and the changes in the items (1) to (6) with time until Week 16 after the beginning of the rearing on the CDAA diet substantially free of protein, in each individual are shown in Table 3.

TABLE 3

| | | | 2 weeks | 4 weeks | 6 weeks | 8 weeks | 10 weeks | 12 weeks | 14 weeks | 16 weeks |
|---|---|---|---|---|---|---|---|---|---|---|
| | | | after | after | after | after | after | after | after | after |
| | Individual | Standard | beginning | beginning | beginning | beginning | beginning | beginning | beginning | beginning |
| | No. | value | of feeding | of feeding | of feeding | of feeding | of feeding | of feeding | of feeding | of feeding |
| Albumin | 1 | 4.248 | 4.105 | 3.851 | 3.622 | 3.206 | 2.638 | 2.206 | 1.853 | 1.786 |
| (mg/dl) | 2 | 4.047 | 4.065 | 3.966 | 3.428 | 3.067 | 2.866 | 2.166 | 1.533 | 1.467 |
| | 3 | 4.188 | 4.024 | 3.824 | 3.610 | 3.168 | 2.795 | 2.083 | 1.428 | 1.420 |
| | 4 | 4.255 | 4.166 | 3.764 | 3.428 | 3.022 | 2.619 | 2.185 | 1.622 | 1.591 |
| | 5 | 4.173 | 4.032 | 3.820 | 3.326 | 3.153 | 2.547 | 2.179 | 1.582 | 1.316 |
| | 6 | 4.206 | 4.027 | 3.751 | 3.482 | 3.088 | 2.528 | 2.248 | 1.671 | 1.583 |
| | Average | 4.186 | 4.070 | 3.829 | 3.483 | 3.117 | 2.666 | 2.177 | 1.615 | 1.527 |
| | value | | | | | | | | | |
| Cholesterol | 1 | 204 | 187 | 154 | 126 | 116 | 105 | 98 | 85 | 83 |
| (mg/dl) | 2 | 217 | 196 | 152 | 128 | 106 | 99 | 94 | 84 | 80 |
| | 3 | 218 | 182 | 146 | 116 | 107 | 106 | 96 | 86 | 83 |
| | 4 | 224 | 179 | 139 | 124 | 112 | 100 | 97 | 76 | 75 |
| | 5 | 224 | 174 | 146 | 118 | 109 | 100 | 94 | 80 | 74 |
| | 6 | 205 | 174 | 142 | 129 | 112 | 99 | 85 | 77 | 74 |
| | Average | 215 | 182 | 147 | 124 | 110 | 102 | 94 | 81 | 78 |
| | value | | | | | | | | | |
| Hepaplastin test | 1 | 224 | 197 | 156 | 128 | 106 | 98 | 81 | 72 | 71 |
| (HPT) value | 2 | 216 | 203 | 146 | 131 | 110 | 100 | 80 | 70 | 69 |
| (%) | 3 | 221 | 196 | 152 | 125 | 108 | 99 | 81 | 69 | 67 |
| | 4 | 206 | 195 | 157 | 132 | 110 | 98 | 81 | 75 | 72 |
| | 5 | 221 | 201 | 162 | 135 | 120 | 98 | 80 | 70 | 65 |
| | 6 | 221 | 198 | 153 | 126 | 105 | 96 | 83 | 71 | 68 |
| | Average | 218 | 198 | 154 | 130 | 110 | 98 | 81 | 71 | 69 |
| | value | | | | | | | | | |
| Type IV collagen | 1 | 0.16 | 0.13 | 0.15 | 0.28 | 0.31 | 0.38 | 0.57 | 0.83 | 0.91 |
| (μg/ml) | 2 | 0.04 | 0.1 | 0.12 | 0.25 | 0.31 | 0.41 | 0.59 | 0.92 | 0.99 |
| | 3 | 0.06 | 0.09 | 0.14 | 0.23 | 0.28 | 0.4 | 0.6 | 0.95 | 1.08 |
| | 4 | 0.12 | 0.17 | 0.19 | 0.27 | 0.33 | 0.37 | 0.65 | 0.85 | 0.98 |
| | 5 | 0.08 | 0.13 | 0.15 | 0.21 | 0.37 | 0.39 | 0.71 | 0.91 | 1.28 |
| | 6 | 0.11 | 0.29 | 0.22 | 0.29 | 0.33 | 0.41 | 0.58 | 0.64 | 0.75 |
| | Average | 0.095 | 0.15 | 0.16 | 0.26 | 0.32 | 0.39 | 0.62 | 0.85 | 1.00 |
| | value | | | | | | | | | |
| Alanine | 1 | 37 | 46 | 59 | 82 | 117 | 157 | 185 | 216 | 287 |
| aminotransferase | 2 | 39 | 51 | 61 | 73 | 108 | 162 | 184 | 251 | 296 |
| (ALT) | 3 | 35 | 48 | 65 | 88 | 96 | 148 | 195 | 208 | 255 |
| (U/l) | 4 | 30 | 39 | 44 | 78 | 110 | 161 | 184 | 225 | 276 |
| | 5 | 28 | 45 | 48 | 83 | 121 | 150 | 174 | 261 | 352 |
| | 6 | 32 | 46 | 53 | 82 | 115 | 166 | 192 | 241 | 281 |
| | Average | 34 | 46 | 55 | 81 | 111 | 157 | 186 | 234 | 291 |
| | value | | | | | | | | | |
| Hyaluronic acid | 1 | 36 | 34 | 31 | 30 | 37 | 39 | 55 | 63 | 71 |
| (ng/ml) | 2 | 32 | 32 | 30 | 34 | 38 | 37 | 51 | 58 | 62 |
| | 3 | 30 | 31 | 33 | 36 | 33 | 36 | 42 | 54 | 60 |
| | 4 | 28 | 26 | 31 | 33 | 35 | 39 | 58 | 61 | 69 |
| | 5 | 32 | 28 | 33 | 37 | 32 | 35 | 50 | 59 | 68 |
| | 6 | 26 | 27 | 29 | 27 | 30 | 35 | 49 | 59 | 61 |
| | Average | 31 | 30 | 31 | 33 | 34 | 37 | 51 | 59 | 65 |
| | value | | | | | | | | | |

### (c) Comparison of Measurement Results

Table 4 shows the results of comparison of each of the items (1) to (6) between the measured values at the time when the pigs were reared (acclimated) on the normal diet MP-A for about 1 week (standard values), and the measured values obtained every 2 weeks until Week 16 after feeding with the CDAA diet substantially free of protein.

TABLE 4

| | 2 weeks after | 4 weeks after | 6 weeks after | 8 weeks after | 10 weeks after | 12 weeks after | 14 weeks after | 16 weeks after |
|---|---|---|---|---|---|---|---|---|
| | beginning of | beginning of | beginning of | beginning of | beginning of | beginning of | beginning of | beginning of |
| Itme | feeding | feeding | feeding | feeding | feeding | feeding | feeding | feeding |
| Blood albumin level | — | — | ↓ | ↓ | ↓ ↓ | ↓ ↓ | ↓ ↓ ↓ | ↓ ↓ ↓ |
| Total blood | ↓ | ↓ ↓ | ↓ ↓ | ↓ ↓ | ↓ ↓ ↓ | ↓ ↓ ↓ | ↓ ↓ ↓ | ↓ ↓ ↓ |
| cholesterol level | | | | | | | | |
| Hepaplastin test | — | ↓ | ↓ ↓ | ↓ ↓ | ↓ ↓ ↓ | ↓ ↓ ↓ | ↓ ↓ ↓ | ↓ ↓ ↓ |
| (HPT) value | | | | | | | | |
| Blood type IV | ↑ ↑ ↑ | ↑ ↑ ↑ | ↑ ↑ ↑ | ↑ ↑ ↑ | ↑ ↑ ↑ | ↑ ↑ ↑ | ↑ ↑ ↑ | ↑ ↑ ↑ |
| collagen level | | | | | | | | |
| Blood alanine | ↑ ↑ | ↑ ↑ ↑ | ↑ ↑ ↑ | ↑ ↑ ↑ | ↑ ↑ ↑ | ↑ ↑ ↑ | ↑ ↑ ↑ | ↑ ↑ ↑ |
| aminotransferase | | | | | | | | |
| (ALT) level | | | | | | | | |
| Blood hyaluronic | — | — | — | — | ↑ | ↑ ↑ ↑ | ↑ ↑ ↑ | ↑ ↑ ↑ |
| acid level | | | | | | | | |

In Table 4, the upward arrows and downward arrows represent increases and decreases, respectively, in the average measured value relative to the average standard value in the six individuals. Each of a single arrow, double arrow, and triple arrow indicates an increase or decrease in the average measured value by not less than 10%, by not less than 30%, and by not less than 50%, respectively, relative to the average standard value. Each hyphen indicates an increase or decrease by less than 10%.

As can be seen from the results shown in Tables 3 and 4, all six individuals of micromini pigs reared on the CDAA diet substantially free of protein showed remarkable decreases in the blood albumin level, total cholesterol level, and hepaplastin test (HPT) value. On the other hand, they showed remarkable increases in the blood type IV collagen level, alanine aminotransferase (ALT) level, and hyaluronic acid level.

### (d) Biopsy of Liver Tissue

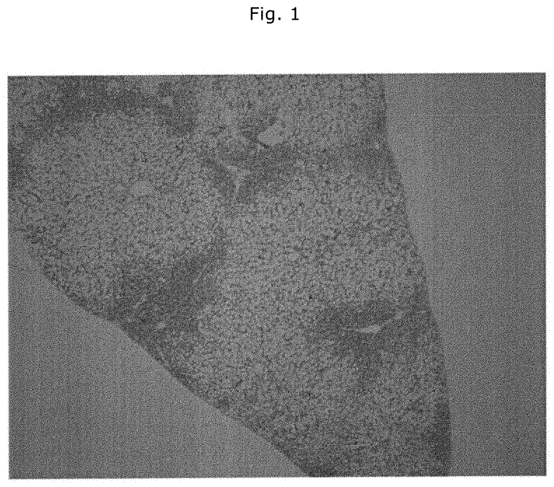

From one individual (Individual Number: 3) out of the six individuals of micromini pigs, a liver tissue was removed, and a biopsy photograph was obtained therefrom. The biopsy photograph is shown in FIG. 1.

In the biopsy photograph shown in FIG. 1, the gray areas correspond to hepatocytes, and the white small granular areas correspond to lipid (lipid droplets). The micromini-pig liver tissue obtained by the method of the present invention showed deposition of a large amount of lipid (lipid droplets) in hepatocytes, which is not found in normal liver tissues. Thus, NAFLD was found also from the biopsy photograph.

From these results, it became clear that micromini pigs reared on the CDAA diet substantially free of protein exhibit clinical manifestations of NAFLD at Week 6 to Week 12 after the beginning of feeding with the diet. In particular, at Week 10 to Week 12 after the beginning of feeding, they reflected most part of the disease state characteristic to human NAFLD. Thus, it could be confirmed that an NAFLD model animal which can be extrapolated to human NAFLD can be prepared by feeding with the CDAA diet substantially free of protein. Mice reared on such a CDAA diet substantially free of protein showed slow progression of the disease state similarly to humans with NAFLD.

Thus, NAFLD model animals of the present invention are thought to be suitable for analysis of the disease state of NAFLD. It is also thought that a CDAA diet substantially free of protein can be used for inducing non-alcoholic fatty liver disease (NAFLD) in non-human animals.

### INDUSTRIAL APPLICABILITY

By the present invention, a non-human model animal for non-alcoholic fatty heart disease (NAFLD) which exhibits clinical manifestations similar to those in humans can be prepared. By utilizing the non-human model animal, development of a therapeutic method and a therapeutic agent for human NAFLD is possible

1. A method of preparing a non-human model animal for non-alcoholic fatty liver disease (NAFLD), the method comprising the step of rearing a non-human animal by feeding the animal with a choline-deficient L-amino acid-defined (CDAA) diet,
wherein the CDAA diet is substantially free of protein.

2. The method according to claim 1, wherein the non-human animal is an animal belonging to a mammalian order selected from the group consisting of Primates, Rodentia, Carnivora, and Artiodactyla.

3. The method according to claim 1, wherein the non-human animal is a pig.

4. A non-human model animal for non-alcoholic fatty liver disease (NAFLD) prepared by the method according to claim 1.

5. A non-human animal model for non-alcoholic fatty liver disease (NAFLD), which exhibits at least one of the following findings (1) to (6):
(1) a decrease in the total blood cholesterol level;
(2) a decrease in the blood albumin level;
(3) a decrease in the hepaplastin test (HPT) value;
(4) an increase in the blood type IV collagen level;
(5) an increase in the blood alanine aminotransferase (ALT) level; and
(6) an increase in the blood hyaluronic acid level.

6. A choline-deficient L-amino acid-defined (CDAA) diet which is substantially free of protein, to be used for causing non-alcoholic fatty liver disease (NAFLD) in a non-human animal.
